(19) United States
(12) Patent Application Publication
(10) Pub. No.: US 2004/0170632 A1
(43) Pub. Date: Sep. 2, 2004

(54) Liquid formulation comprising cetuximab and a fatty acid ester of polyoxyethylene sorbitan

(76) Inventors: Hanns Christian Mahler, Wiesbaden (DE); Robert Muller, Darmstadt (DE); Ulrike Martini-Marr, Pfungstadt (DE); Udo Haas, Darmstadt (DE); Christiane Bachmann, Goldbach (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/483,404

(22) PCT Filed: Jun. 18, 2002
(86) PCT No.: PCT/EP02/06696

(30) Foreign Application Priority Data
Jul. 13, 2001 (DE) 10133394.3

(51) Int. Cl. A61K 39/395

(52) U.S. Cl. 424/155.1

(57) ABSTRACT

The invention relates to a stable liquid pharmaceutical formulation comprising Cetuximab®, a chimeric monoclonal antibody against the receptor of endothelial growth factor (EGF receptor). The formulation has increased storage stability and can be used parenterally for the treatment of tumours.

The present invention relates to a stable liquid pharmaceutical formulation comprising the chimeric monoclonal antibody C225 (Cetuximab®) against the receptor of epidermal growth factor (EGF receptor).

Various in vitro and in vivo studies have shown that blockage of the EGF receptor by antibodies act against tumours on various levels, for example by inhibiting cancer cell proliferation, reducing tumour-mediated angio-genesis, inducing cancer cell apoptosis and increasing the toxic effects of radiotherapy and conventional chemotherapy. Cetuximab® is a highly promising antibody which binds to the EGF receptor. Cetuximab® or C225 is recombined from the DNA of various species and was described for the first time by Naramura et al. (Cancer Immunol. Immunotherapy 37, 343-349, 349, 1993). With regard to the preparation of Cetuximab®, reference is made to the said scientific literature.

Like other antibodies, Cetuximab® is applied parenterally as a solution for therapeutic application. A particular problem of solutions containing antibodies is their tendency towards aggregation and towards the formation of protein multimers. In the case of reducible multimers, this can be attributed to unintentional intermolecular disulfide bridge formation through an interaction between adjacent moieties. Hydrophobic interactions and the associated formation of non-reducible multimers are also possible. Furthermore, deamidation reactions occur, resulting in subsequent protein degradation reactions.

As a consequence of the said tendency toward aggregation, product precipitations occur on storage of antibody solutions, and consequently reproducible removal from the container containing the solution is put in doubt. In addition, embolisms can form on parenteral application of particle-containing solution. This has the consequence that reproducible administration of the dose necessary in each case to the patient is not guaranteed and the application cannot take place with the requisite safety. Although the aggregates can be held back by filtration before injection, this method entails, however, an additional step and is therefore complex and not very suitable for clinical practice. The problem of dose reproducibility also remains unsolved, since an unknown proportion of antibodies is in each case separated off from the solution and particle formation after filtration continues to represent a safety risk.

A common method for stabilising monoclonal antibodies is freeze-drying of solutions containing antibodies and auxiliaries. However, lyophilisation is very time- and energy-consuming and consequently expensive. The lyophilisate also has to be reconstituted before administration.

EP 0 073 371 describes immunoglobulin compositions which can be adminlistered intravenously and, for stabilisation, have a pH of from 3.5 to 5.0. However, such low pH values result in undesired intolerance reactions at the site of injection.

U.S. Pat. No. 6,171,586 B1 discloses the use of an acetate buffer pH 4.48 to 5.5, a surfactant and a polyol in a liquid formulation of antibodies, with NaCl being excluded for establishing isotonicity. Owing to the low pH and the lack of isotonicity, intolerance reactions at the site of injection can likewise occur.

As examples of further formulations comprising specific antibodies, mention may be made at this point of EP 0 280 358, EP 0 170 983 and U.S. Pat. No. 5,945,098.

Of these, EP 0 280 358 describes the addition of dextran to an antibody solution for stabilisation against certain hormones, with stability being achieved over nine months.

EP 0 170 983 describes the stabilisation of a thermally labile monoclonal antibody by heating together with hydrolysed ovalbumin, causing the antibody to remain stable after storage for 7 days at 45° C. However, the addition of proteins from other species to administerable formulations intended for parenteral administration are undesired owing to the problems associated therewith, in particular their possible antigeneity.

U.S. Pat. No. 5,945,098 discloses the use of glycine, polysorbate 80 and polyethylene glycol for the stabilisation of a liquid formulation of immunoglobulin G.

The object of the invention was to find especially for Cetuximab® a liquid formulation which is suitable for parenteral administration, is well tolerated and is stable for at least one year on storage at room temperature. The formulation should have a simple composition and should not comprise any auxiliaries which are questionable from a toxicological point of view.

Surprisingly, a formulation which meets these requirements has been found in the form of a solution which, besides Cetuximab®, comprises a phosphate buffer in the range from about pH 6 to about pH 8 and a polyoxyethylene sorbitan fatty acid ester. The present invention therefore relates to a stable liquid pharmaceutical composition which comprises a phosphate buffer in the range from pH 6 to pH 8 and a polyoxyethylene sorbitan fatty acid ester. The pH is preferably in the range from 6.5 to 7.5, a pH of about 7.2 being particularly preferred.

Phosphate buffers which can be employed are solutions of the mono- and/or disodium and -potassium salts of phosphoric acid, such as disodium hydrogenphosphate or potassium dihydrogenphosphate, and mixtures of the sodium and potassium salts, such as, for example, mixtures of disodium hydrogenphosphate and potassium dihydrogenphosphate. The phosphate buffer may be present in the formulation according to the invention in a concentration range from 2 mM to 100 mM. Preference is given to a concentration range from 5 mM to 20 mM, particularly preferably about 10 mM.

Cetuximab® may be present in the formulation according to the invention in a concentration of from 0.1 mg/ml to 25 mg/ml. Preferably from 2 mg/ml to 10 mg/ml, particularly preferably about 5 mg/ml, are present.

Polyethylene sorbitan fatty acid esters are also known under the trade name Tween. The formulation according to the invention may comprise, in particular, polyoxyethylene (20) sorbitan monolaurate, polyoxyethylene (20) sorbitan monopalmitate and polyoxyethylene (20) sorbitan monostearate. Preference is given to polyoxyethylene (20) sorbitan monolaurate and polyoxyethylene (20) sorbitan monooleate, of which particular preference is given to polyoxyethylene (20) sorbitan monooleate. The polyethylene sorbitan fatty acid esters may be present in the formulation in a concentration of from 0.001% to 1.0%. Preferably from 0.005% to 0.1%, particularly preferably about 0.01%, are present.

The formulation according to the invention advantageously additionally comprises an isotonic agent, preferably a physiologically tolerated salt, such as, for example, sodium chloride or potassium chloride, or a physiologically tolerated polyol, such as, for example, glucose or glycerol, in a concentration necessary for establishing isotonicity. The invention therefore relates to a liquid formulation comprising Cetuximab®, a phosphate buffer in the range from about pH 6 to about pH 8, a polyoxyethylene sorbitan fatty acid ester and an isotonic agent in a concentration necessary for establishing isotonicity. The formulation preferably comprises sodium chloride as isotonic agent.

According to a particularly advantageous embodiment of the invention, the liquid formulation comprises about 5 mg/ml of Cetuximab®, about 10 mM of phosphate buffer having a pH of about 7.2, about 145 mM of sodium chloride and about 0.01% of polyoxyethylene (20) sorbitan monooleate.

The formulation according to the invention can be prepared by adding the said constituents to a Cetuximab®-containing solution. To this end, defined volumes of stock solutions comprising the said further constituents in defined concentration are advantageously added to a solution having a defined concentration of Cetuximab® as obtained in the preparation of the latter, and the mixture is, where appropriate, diluted with water to the pre-calculated concentration. Alternatively, the constituents can also be added to the Cetuximab®-containing starting solution as solids. If Cetuximab® is in the form of a solid, for example in the form of a lyophilisate, the formulation according to the invention can be prepared by firstly dissolving Cetuximab® in water or an aqueous solution comprising one or more of the further constituents, and subsequently adding the amounts necessary in each case of stock solutions comprising the further constituents, the further constituents in solid form and/or water. Cetuximab® may advantageously also be dissolved directly in a solution comprising all further constituents. One or more of the constituents present in the formulation according to the invention may advantageously be added as early as during or at the end of the Cetuximab® preparation process. This can preferably be carried out by dissolving Cetuximab® directly in an aqueous solution comprising one, several or all further constituents in the final step of the purification carried out after its preparation. In order to prepare the formulation, the respective further constituent(s) then only have to be added in a smaller amount in each case and/or not added at all. It is particularly preferred if the respective constituent is dissolved directly in an aqueous solution comprising all further constituents in the final step of the purification carried out after its preparation, so that the formulation according to the invention is obtained directly.

The examples, without being restricted thereto, explain the invention.

### EXAMPLE 1

Aqueous solution comprising:

5 mg/ml of Cetuximab®

10 mM of sodium phosphate buffer pH 7.2

45 mM of sodium chloride

0.01% by weight of polyoxyethylene (20) sorbitan monooleate.

The preparation was carried out by mixing defined volumes of aqueous solutions comprising the respective constituents in defined concentration. The following solutions were used:

Solution A (active ingredient solution) comprising:

9.7 mg/ml of Cetuximab®

10 mM of sodium phosphate buffer pH 7.2 (consisting of 2.07 g/l of disodium hydrogenphosphate 7-hydrate and 0.31 g/l of sodium dihydrogen-phosphate monohydrate)

145 mM of sodium chloride.

(The solution was obtained by eluting the active ingredient from the column with solution B in the final step of the chromatographic active ingredient purification carried out after its preparation.)

Solution B (buffer/salt solution):

corresponds to solution A, but comprises no active ingredient.

Solution C (polyoxyethylene sorbitan fatty acid ester solution):

corresponds to solution B, but additionally comprises 1% by weight of polyoxyethylene (20) sorbitan monooleate.

In order to prepare the formulation according to the invention, 10 ml of solution A, 9.8 ml of solution B and 0.2 ml of solution C were combined with one another.

The prepared solution was filtered using a sterile filter before transfer into vials. The vials were each filled with 2 ml of solution using a pipette. The vials were subsequently sealed with stoppers and crimped.

### EXAMPLE 2 (Comparative Formulation)

Aqueous solution comprising:

5 mg/ml of Cetuximab®

10 mM of sodium phosphate buffer pH 7.2

145 mM of sodium chloride

In order to prepare the comparative formulation, 10 ml of each of solutions A and B described in Example 1 were combined with one another.

### EXAMPLE 3

Aqueous solution comprising:

2 mg/ml of Cetuximab®

0.1% by weight of polyoxyethylene (20) sorbitan monolaurate

20 mM of disodium hydrogenphosphate

5% by weight of glucose

The preparation was carried out by mixing defined volumes of aqueous solutions comprising the respective constituents in defined concentration. The following solutions were used:

Solution A:

Aqueous solution comprising:

4 mg/ml of Cetuximab®

20 mM of disodium hydrogenphosphate

(The solution was obtained by eluting the active ingredient from the column with solution B in the final step of the chromatographic active ingredient purification carried out after its preparation.)

Solution B (polyoxyethylene sorbitan fatty acid ester/glucose solution):

0.2% by weight of polyoxyethylene (20) sorbitan monolaurate

10% by weight of glucose

20 mM of disodium hydrogenphosphate

For the preparation, 10 ml of solution A and 10 ml of solution B were combined with one another.

The prepared solution was filtered using a sterile filter before transfer into vials. The vials were each filled with 2 ml of solution using a pipette. The vials were subsequently sealed with stoppers and crimped.

### EXAMPLE 4

The stability of the formulation according to the invention was tested in a stress test. To this end, vials containing the solution according to Example 1 and, for comparative purposes, vials containing solution according to

Example 2 were stored at 40° C. and 75% relative atmospheric humidity. Before storage and after defined storage times, in each case 3 vials were assessed visually under direct illumination with a cold light source, and the absorption of the solutions at 350 and 550 nm, which represents a measure of the cloudiness, was determined. Furthermore, 3 vials were removed in each case and analysed with regard to the content of Cetuximab® and decomposition products by means of HPLC gel filtration.

In the gel filtration HPLC, phosphate buffer pH 7.2 was employed as mobile medium. Column: Toso Haas TSKgel G 3000 SWXL (ID 7.8 mm, length 30 cm), flow rate: 0.5 ml/min. The detection was carried out at 280 nm.

The results of the stability studies are shown in Table 1.

TABLE 1

| | | | Sec. | Decomposition | Cloudiness | Cloudiness | |
|---|---|---|---|---|---|---|---|
| Test | Storage | Cetuximab | zones | products | at | at | Visual |
| solution | [weeks] | [%] | [%] | [%] | λ = 350 nm | λ = 550 nm | assessment |
| Example 1 | 0 | 99.72 | 0.11 | 0.17 | 0.0128 | 0.0016 | clear |
| Example 1 | 4 | 98.60 | 0.84 | 0.56 | 0.0200 | 0.0022 | clear |
| Example 1 | 8 | 96.49 | 1.30 | 2.21 | 0.0280 | 0.0033 | clear |
| Example 2 | 0 | 99.69 | 0.15 | 0.16 | 0.0130 | 0.0021 | clear |
| Example 2 | 4 | 92.00 | 7.38 | 0.62 | 0.0232 | 0.0047 | small |
| | | | | | | | particles |

The results clearly show that the formulation according to the invention has significantly increased stability compared with the comparative solution.

1. Liquid pharmaceutical formulation comprising Cetuximab®, a phosphate buffer pH 6 to pH 8 and a polyoxyethylene sorbitan fatty acid ester.

2. Liquid pharmaceutical formulation according to claim 1, characterised in that this has a pH of from pH 6.5 to pH 7.5.

3. Liquid pharmaceutical formulation according to claim 2, characterised in that this has a pH of about pH 7.2.

4. Liquid pharmaceutical formulation according to one or more of claims 1 to 3, characterised in that the phosphate buffer is present in a concentration of from 2 mM to 100 mM.

5. Liquid pharmaceutical formulation according to claim 4, characterised in that the phosphate buffer is present in a concentration of from 5 mM to 20 mM, preferably 10 mM.

6. Liquid pharmaceutical formulation according to one or more of claims 1 to 5, characterised in that the polyoxyethylene sorbitan fatty acid ester present is polyoxyethylene (20) sorbitan monooleate or polyoxyethylene (20) sorbitan monolaurate.

7. Liquid pharmaceutical formulation according to one or more of claims 1 to 6, characterised in that the polyoxyethylene sorbitan fatty acid ester is present in a concentration of from 0.005% to 0.1%, in particular in a concentration of about 0.01%.

8. Liquid pharmaceutical formulation according to one or more of claims 1 to 7, characterised in that an isotonic agent is furthermore present in a concentration necessary for establishing isotonicity.

9. Liquid pharmaceutical formulation according to claim 8, characterised in that sodium chloride is present as isotonic agent.

10. Liquid pharmaceutical formulation according to one or more of claims 1 to 9, characterised in that this comprises about 5 mg/ml of Cetuximab®, about 10 mM of phosphate buffer having a pH of about 7.2 , about 145 mM of sodium chloride and about 0.01% of polyoxyethylene (20) sorbitan monooleate.
